(19) United States
(12) Patent Application Publication
(10) Pub. No.: US 2011/0111515 A1
(43) Pub. Date: May 12, 2011

(54) REAGENT FOR MEASUREMENT OF REACTIVE OXYGEN

(75) Inventors: Tetsuo Nagano, Tokyo (JP); Hirotatsu Kojima, Tokyo (JP); Daihi Oushiki, Tokyo (JP)

(73) Assignee: THE UNIVERSITY OF TOKYO, Tokyo (JP)

(21) Appl. No.: 12/919,059

(22) PCT Filed: Feb. 27, 2009
(86) PCT No.: PCT/JP2009/053658

§ 371 Date: Jan. 25, 2011

(30) Foreign Application Priority Data
Feb. 29, 2008 (JP) 2008-049651

(51) Int. Cl. G01N 33/00; C07D 209/04

(52) U.S. Cl. 436/116; 548/455; 436/127

(57) ABSTRACT

A reagent for measurement of reactive oxygen, which can be used with a light of the near infrared region showing superior biological tissue permeability, wherein (i) a first cyanine compound residue and a second cyanine compound residue are bound to each other, (ii) the first cyanine compound residue has a property that it easily reacts with a reactive oxygen species and is thereby decomposed, and (iii) the second cyanine compound residue either equals or surpasses the first cyanine compound residue in its stability to the reactive oxygen species, and the first cyanine compound residue acts as a quenching group for the second cyanine compound residue.

### TECHNICAL FIELD

The present invention relates to a reagent for measurement of reactive oxygen, which consists of two cyanine compound residues bound via a linker.

### BACKGROUND ART

It has been reported that reactive oxygen species are playing various important roles in living bodies. For example, nitrogen monoxide is known to act as a second messenger of signal transduction, and exert various physiological actions such as an action of controlling blood pressure in the circulatory system. It has been shown that superoxide anions and hydrogen peroxide exert important physiological actions in the immune system, and the like. Many findings have been reported for involvement of hydroxyl radicals in angiopathy, brain disorders after ischemia, and DNA modification by ultraviolet radiation, and hydroxyl radical is considered to be an especially highly obstructive reactive oxygen species in connection with etiology and pathology.

Peroxynitrite (ONOO⁻), which is generated by the reaction of nitrogen monoxide and a superoxide anion, has strong oxidizing power, for example, it enables nitration of an aromatic ring, and shows characteristic reactivity, for example, it achieves efficient nitration of tyrosine. A latest report has pointed out that nitration of tyrosine inhibits phosphorylation of tyrosine to significantly effect signal transduction systems such as the MAPK and PI3 K/Akt cascades. Furthermore, the actions of hypochlorite in living bodies have been focused in recent years. It is considered that the bactericidal action of neutrophiles is mainly based on hypochlorite ion, and it has been demonstrated in vitro that hypochlorite ion is generated from hydrogen peroxide and chloride ion by myeloperoxidase in the azurophil granules (Klebanoff, S. J. et al., The Neutrophils: Function and Clinical Disorders, North-Holland Publishing Company, Amsterdam, Netherlands, 1978). It has also been reported that hypochlorite ion plays an important role in injury of the vascular endothelium surface in microcirculation dysfunction induced by the platelet activating factor (Suematsu, M., et al., J. Biochem., 106, pp. 355-360, 1989).

Since reactive oxygen species are involved in various diseases such as inflammation, senility, and arteriosclerosis, and signal transduction as described above, importance of elucidating the roles of various reactive oxygen species in the living bodies is increasing, and several fluorescent probes for measuring reactive oxygen species in the living bodies have been proposed. For example, there are known the reactive oxygen fluorescent probe described in International Patent Publication WO01/64664 (J. Biol. Chem., 278, pp. 3170-3175, 2003), singlet oxygen fluorescent probes described in International Patent Publications WO99/51586 and WO02/18362, nitrogen monoxide fluorescent probes described in Japanese Patent Laid-Open Publications (Kokai) No. 10-226688 and International Patent Publication WO2004/76466, H₂DCFDA (2′,7′-dichlorodihydro-fluorescein diacetate, Molecular Probe, catalog number: D-399), and the like. There have also been proposed a method of measuring superoxide anions (Clinica Chimica Acta, 179, pp. 177-182, 1989) or singlet oxygen (J. Biolumin. Chemilumin., 6, pp. 69-72, 1991) by a chemiluminescence method using a cypridina luciferin derivative, MCLA, a method of measuring reactive oxygen species using a luciferin derivative as a bioluminescence probe for reactive oxygen species (International Patent Publication WO2007/111345), and the like. However, many of these fluorescent probes have absorption and fluorescence (emission) wavelengths in the visible light region, of which lights show low biological tissue permeability, and therefore they are not probes which enable in vivo visualization of reactive oxygen species.

In recent years, imaging techniques utilizing a probe having absorption and fluorescence wavelengths in the near infrared region of around 650 to 950 nm as a fluorescent probe for non-invasively imaging biological phenomena have been focused in the field of life chemical researches. For example, carbocyanine dyes show maximum absorption wavelength and maximum fluorescence wavelength in the near infrared region of around 650 to 950 nm, lights of which range are comparatively less absorbed by biological molecules, and therefore they have an advantage in that they enable use of light of a wavelength which can penetrate into deep parts of biological tissues. In addition, biological substances show less autofluorescence in the near infrared region. More specifically, the characteristics of carbocyanine dyes are preferable for in vivo imaging. In addition to the cyanine dyes for directly labeling biological molecules with fluorescence, carbocyanine dyes showing change of fluorescence intensity by specifically reacting with a biological molecule have recently been developed. One aspect is the near-infrared fluorescent probe for calcium ion (Ozmen, B., et al., Tetrahedron Lett., 41, pp. 9185-9188, 2000), and another aspect is the near-infrared fluorescent probe for nitrogen monoxide (NO) (International Patent Publication WO2005/080331). These fluorescent probes are probes showing only change of fluorescence intensity without change of excitation/fluorescence wavelengths before and after a specific reaction with a biological molecule.

The inventors of the present invention proposed a tricarbocyanine type fluorescent probe which enables imaging of zinc ion concentration by the ratio method (International Patent Publication WO2005/080331) and a tricarbocyanine type fluorescent probe which enables imaging of pH by the ratio method (International Patent Publication WO2008/099914). These probes are ratio fluorescent probes of which excitation wavelengths shift depending on change of zinc ion concentration or pH. The inventors of the present invention also proposed a tricarbocyanine type fluorescent probe for pH measurement, which utilized fluorescence change induced by fluorescence resonance energy transfer (FRET) (International Patent Publication WO2008/108074). These fluorescent probes based on the ratio method have an advantage that they enable quantitative measurement of measurement object regardless of probe concentration, intensity of light source, size of cells, and the like. Furthermore, probes utilizing tricarbocyanine dyes for various enzymes have also been proposed. There are, for example, the fluorescent probe for protease described in International Patent Publication WO99/58161, the fluorescent probe for β-lactamase described in J. Am. Chem. Soc., 2005, 127, 4158-4159, the fluorescent probe for cysteine protease described in Nat. Chem. Biol., 2007, 10, 668-677, and the like. The fluorochromes and the quenching groups of these fluorescent probes for various enzymes are bound via a linker, and the fluorochromes and the quenching groups are cleaved by an enzymatic reaction to form an active fluorescent dye. However, almost no methods of using a carbocyanine dye as a fluorescent probe for reactive oxygen species have been known except for the method of using a fluorescent probe for NO.
Patent document 1: International Patent Publication WO01/64664
Patent document 2: International Patent Publication WO99/51586
Patent document 3: International Patent Publication WO02/18362
Patent document 4: Japanese Patent Laid-Open Publication (Kokai) No. 10-226688
Patent document 5: International Patent Publication WO2004/76466
Patent document 6: International Patent Publication WO2007/111345
Patent document 7: International Patent Publication WO2005/080331
Patent document 8: International Patent Publication WO2008/099914
Patent document 9: International Patent Publication WO2008/108074
Patent document 10: International Patent Publication WO99/58161
Non-patent document 1: Clinica Chimica Acta, 179, pp. 177-182, 1989
Non-patent document 2: J. Biolumin. Chemilumin., 6, pp. 69-72, 1991
Non-patent document 3: J. Am. Chem. Soc., 2005, 127, 4158-4159
Non-patent document 4: Nat. Chem. Biol., 2007, 10, 668-677

### DISCLOSURE OF THE INVENTION

### Object to be Achieved by the Invention

An object of the present invention is to provide a reagent for measurement of reactive oxygen. More specifically, the object of the present invention is to provide a reagent for measurement of reactive oxygen as a fluorescent probe which can utilize a wavelength in the near infrared region, of which light shows superior tissue permeability.

### Means for Achieving the Object

Cyanine compounds are typical dyes widely used for the measurement of fluorescence of the near infrared region. The inventors of the present invention conducted various researches in order to provide a probe that achieves successful measurement of reactive oxygen species based on measurement of fluorescence of the near infrared region using a cyanine compound. Since cyanine compounds have a long conjugated polymethine chain, they have a property that the conjugated polymethine chain easily reacts with reactive oxygen species to induce decomposition of the compounds, and thus they lose absorption and fluorescence thereof in the near infrared region upon the reaction with reactive oxygen species. Therefore, they designed a reagent for measurement of reactive oxygen utilizing that property, i.e., by combining a first cyanine compound residue having a long conjugated polymethine chain as a capturing (reaction) moiety for a reactive oxygen species with a second cyanine compound residue stable to the reactive oxygen species, so that the first cyanine compound residue can act as a quenching group for the second cyanine compound residue. When this reagent was used as a fluorescent probe for measurement of reactive oxygen, it was confirmed that decomposition of the first cyanine compound residue occurred by a reaction with a reactive oxygen species restored fluorescence of the second cyanine compound residue, which enabled the probe to emit strong fluorescence upon irradiation of a light of the near infrared region, and it was confirmed that it had an extremely superior property as a reagent for measurement of reactive oxygen. The present invention was accomplished on the basis of the aforementioned finding.

The present invention thus provides a reagent for measurement of reactive oxygen containing a compound comprising a first cyanine compound residue and a second cyanine compound residue bound to each other and having the following characteristics features (i) to (iii):

(i) the first cyanine compound residue and the second cyanine compound residue are directly bound to each other via substituents on the first cyanine compound residue and the second cyanine compound residue, or the first cyanine compound residue and the second cyanine compound residue are bound via a linker,
(ii) the first cyanine compound residue has a property that it easily reacts with a reactive oxygen species and is thereby decomposed, and
(iii) the second cyanine compound residue either equals or surpasses the first cyanine compound residue in its stability to the reactive oxygen species, and the first cyanine compound residue has a property that it acts as a quenching group for the second cyanine compound residue.

According to a preferred embodiment of the present invention, there is provided the aforementioned reagent, wherein —S— group substitutes for one carbon atom in the conjugated polymethine chain of the first cyanine compound residue, and the second cyanine compound residue has one or two sulfo groups in the nitrogen-containing heterocyclic moiety.

According to preferred embodiments of the present invention, there are further provided the aforementioned reagent, wherein the first cyanine compound residue has the following partial substructure in the fluorophore:

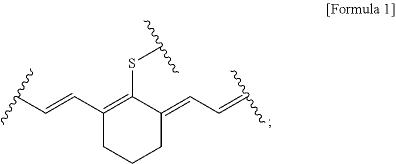

the aforementioned reagent, wherein the second cyanine compound residue has a maximum fluorescence wavelength in the near infrared region, preferably a maximum fluorescence wavelength larger than 650 nm, and shows a fluorescence quantum yield of 0.03 or larger; the aforementioned reagent, wherein the first cyanine compound residue and the second cyanine compound residue are bound via a linker; the aforementioned reagent, wherein the linker binds to carboxy group or sulfo group of the second cyanine compound residue; the aforementioned reagent, wherein the first cyanine compound residue and the second cyanine compound residue are tetramethylindocarbocyanine compound residues; and the aforementioned reagent, wherein linking atomic number of the linker is 4 to 10.

As a particularly preferred embodiment of the aforementioned invention, there is provided a fluorescent probe for measurement of reactive oxygen represented by the following formula as the aforementioned reagent.

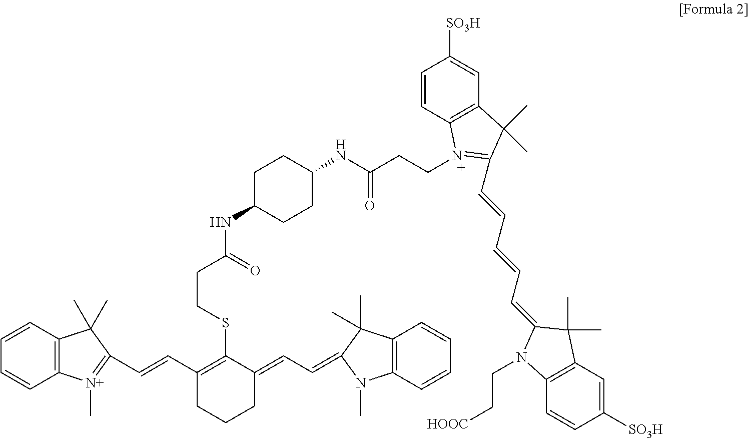

As another aspect of the present invention, there is provided a method for measurement of reactive oxygen species comprising the following steps: (A) reacting the aforementioned reagent and a reactive oxygen species, and (B) measuring fluorescence of a decomposition product of the aforementioned reagent produced in the aforementioned step (A).

### Effect of the Invention

The reagent for measurement of reactive oxygen provided by the present invention has a property that the reagent itself has very weak fluorescent property, whilst it emits strong fluorescence in the near infrared region after reacting with various reactive oxygen species. Therefore, the reagent has a superior characteristic that it enables highly sensitive in vivo measurement of reactive oxygen species without damaging cells or tissues.

### BRIEF DESCRIPTION OF THE DRAWINGS

FIG. 1 shows UV spectra and fluorescence spectra of Compound 2 (cyanine compound constituting the second cyanine compound residue) and Compound 3 (cyanine compound constituting the first cyanine compound residue) obtained in Example 1 among the examples.

FIG. 2 shows results of measurement of change of absorbance at maximum absorption wavelengths after reaction with hydroxyl radical, peroxynitrite, hypochlorite ion, or superoxide anion, performed for Cy5, Cy7, Compound 2 obtained in Example 1 among the examples, and Compound 3 obtained in Example 1 among the examples.

FIG. 3 shows results of reactions of the reagent for measurement of reactive oxygen of the present invention and various kinds of reactive oxygen species. Among the graphs, (a), (b), (c), (d), (e) and (f) show results of reactions with hydroxyl radical, peroxynitrite, hypochlorite ion, superoxide anion, singlet oxygen, and hydrogen peroxide, respectively.

FIG. 4 shows results of measurement of superoxide anions produced by HL60 cells after addition of PMA using the reagent for measurement of reactive oxygen of the present invention.

### BEST MODE FOR CARRYING OUT THE INVENTION

For the reagent of the present invention, it is necessary to choose, as the first cyanine compound residue, a cyanine compound residue having a property that it easily reacts with a reactive oxygen species and is thereby decomposed, and functioning as a quenching group for the second cyanine compound residue. In this specification, a “cyanine compound residue” means a monovalent group produced by eliminating one hydrogen atom of a cyanine compound (for example, carbocyanine compounds, thiacarbocyanine compounds and tetramethylindocarbocyanine compounds; henceforth these may be collectively referred to as carbocyanine compounds). The property of the cyanine compound residue that it easily reacts with a reactive oxygen species, and is thereby decomposed can be determined on the basis of degree of decomposition of the dye measured by, for example, the Fenton reaction, which is widely used as a standard method for generating hydroxyl radicals (.OH), one of the reactive oxygen species. For example, 1 M aqueous hydrogen peroxide (H₂O₂) is added to a final concentration of 1 mM to a 10 μM solution of a cyanine compound in a phosphate buffer (0.1 M, pH 7.4) while vigorously stirred in a flask, and 10 mM aqueous iron(II) is added dropwise to the mixture to a final concentration of 50 μM. Absorbance values at the absorption maximum wavelength of the cyanine compound measured before and after performing this operation are compared, and reactivity of the compound to reactive oxygen species can be defined on the basis of presence or absence of reduction of the absorbance. For example, when 20% or more of the compound is decomposed within 1 minute by the Fenton reaction at 37° C., it can be judged that the compound easily reacts with reactive oxygen species and is thereby decomposed. It is sufficient for the first cyanine compound residue to either equal or surpass the second cyanine compound residue in its reactivity to reactive oxygen species, and it is preferred that the second cyanine compound residue is substantially stable to reactive oxygen species. “Substantially stable to reactive oxygen species” used herein means not only that the residue does not react (to be decomposed or modified) with reactive oxygen species, but also that, even when it reacts with reactive oxygen species, the fluorescent characteristics of the second cyanine residue do not change in the meaning of the relation between the first cyanine compound residue and the second cyanine compound residue.

As the first cyanine compound residue, for example, a cyanine compound residue having the partial structure shown in [Formula 1] above is preferred. More specifically, for example, a residue of a cyanine compound represented by the following general formula (I):

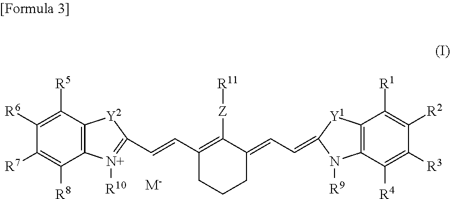

[In the formula, R¹, R², R³, R⁴, R⁵, R⁶, R⁷, and R⁸ independently represent hydrogen atom, sulfo group, phospho group, nitro group, a halogen atom, or a C₁₋₆ alkyl group which may have a substituent; R⁹ and R¹⁰ independently represent a C₁₋₁₈ alkyl group which may have a substituent; R¹¹ represents hydrogen atom or a C₁₋₁₈ alkyl group which may have a substituent; Z represents oxygen atom, sulfur atom, or —N(R¹²)— (wherein R¹² represents hydrogen atom, or a C₁₋₆ alkyl group which may have a substituent, provided that, when Z is —N(R¹²)—, R¹¹ and R¹² do not represent a group which reacts with a reactive oxygen species to affect the fluorescent characteristic of the second cyanine compound residue); Y¹ and Y² independently represent —O—, —S—, or —C(R¹³)(R¹⁴)— (wherein R¹³ and R¹⁴ independently represent a C₁₋₆ alkyl group which may have a substituent); and M⁻ represents a counter ion in a number required for neutralizing the charge] is preferred.

In the specification, the alkyl group may be a linear, branched, or cyclic alkyl group, or a combination thereof, unless otherwise specifically mentioned. When the alkyl group has a substituent, although type, number, and substitution position of the substituent are not particularly limited, it may have, for example, an alkyl group, an alkoxy group, an aryl group, a halogen atom (it may be any of fluorine atom, chlorine atom, bromine atom, and iodine atom), hydroxy group, amino group, nitro group, carboxy group or an ester thereof, sulfo group or an ester thereof, or the like as the substituent.

As the C₁₋₆ alkyl group represented by R¹, R², R³, R⁴, R⁶, R⁶, R⁷, or R⁸, methyl group, ethyl group, and the like are preferred, and as the halogen atom represented by R¹, R², R³, R⁴, R⁸, R⁶, R⁷, or R⁸, fluorine atom, chlorine atom, and the like are preferred. The sulfo group and phospho group represented by R¹, R², R³, R⁴, R⁵, R⁶, R⁷, or R⁸ may form an ester. All of R¹, R², R³, R⁴, R⁵, R⁶, R⁷, and R⁸ may represent hydrogen atom.

Examples of the C₁₋₁₈ alkyl group represented by R⁸, R¹⁰, or R¹¹ include methyl group, ethyl group, n-propyl group, isopropyl group, n-butyl group, isobutyl group, sec-butyl group, tert-butyl group, n-pentyl group, isopentyl group, neopentyl group, tert-pentyl group, 1-methylbutyl group, 2-methylbutyl group, 1-ethylpropyl group, n-hexyl group, 1-methylpentyl group, 2-methylpentyl group, 3-methylpentyl group, 4-methylpentyl group, 2,3-dimethylbutyl group, 1,3-dimethylbutyl group, 1,2-dimethylbutyl group, 1-ethylbutyl group, 2-ethylbutyl group, 1-isopropylpropyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, and the like. As the alkyl group, a linear alkyl group is preferred. Examples of the substituent that can exist on the C₁₋₁₈ alkyl group represented by R⁹ or R¹⁰ include an alkoxy group, an aryl group, a halogen atom (it may be any of fluorine atom, chlorine atom, bromine atom, and iodine atom), hydroxy group, amino group, nitro group, carboxy group or an ester thereof, sulfo group or an ester thereof, and the like. Among these, carboxy group, sulfo group, amino group, and the like are preferred, and carboxy group and sulfo group are particularly preferred. Both of R⁹ and R¹⁰ may represent an unsubstituted C₁₋₁₈ alkyl group, and it is also preferred that one of the C₁₋₁₈ alkyl groups has a substituent. It is preferred that both R⁹ and R¹⁰ represent an unsubstituted alkyl group, and it is more preferred that they both represent methyl group. It is preferred that R¹¹ is a C₁₋₄ alkyl group substituted with carboxy group, and it is preferred that it binds with a linker via this carboxy group. Although bonding scheme with the linker is not particularly limited, examples include an ester bond, an amide bond, and the like. When the first cyanine compound residue and the second cyanine compound residue are directly bound via substituents substituting for the first cyanine compound residue and the second cyanine compound residue, it is preferred that the first cyanine compound residue is bound with the second cyanine compound residue via an ester bond or an amide bond by utilizing carboxy group, sulfo group, amino group or the like substituting for the C₁₋₁₈ alkyl group which may have a substituent represented by R⁹, R¹⁰, or R¹¹.

Z represents oxygen atom, sulfur atom, or —N(R¹²)— (when Z is —N(R¹²)—, R¹¹ and R¹² do not represent a group which reacts with a reactive oxygen species to affect the fluorescent characteristic of the second cyanine compound residue) bound with the linker, and R¹² represents hydrogen atom, or a C₁₋₆ alkyl group which may have a substituent. It is preferred that Z is sulfur atom. When Z is sulfur atom, there is obtained an effect that oxidation potential of the first cyanine compound residue reduces, and reactivity thereof to reactive oxygen species increases. As R¹², hydrogen atom, methyl group, and the like are preferred. Y¹ and Y² independently represent —O—, —S—, or —C(R¹³)(R¹⁴)—, and R¹³ and R¹⁴ independently represent a C₁₋₆ alkyl group which may have a substituent. It is preferred that Y¹ and Y² represent —C(R¹³)(R¹⁴)—, and as R¹³ and R¹⁴, methyl group is preferred. M⁻ represents a counter ion in a number required for neutralizing the charge. Examples of the counter ion include chloride ion, sulfate ion, nitrate ion, perchlorate anion, organic acid anions such as methanesulfonate anion, p-toluenesulfonate anion, oxalate anion, citrate anion, and tartrate anion, ions of amino acids such as glycine, metal ions such as sodium ion, potassium ion and magnesium ion, quaternary ammonium ions, and the like. For example, when carboxy group, sulfo group or the like exists on the C₁₋₁₈ alkyl group represented by R⁹ or R¹⁰ in the general formula (I), or when one or more of R¹, R², R³, R⁴, R⁵, R⁶, R⁷, and R⁸ represent sulfo group or phospho group, and sodium ion is used as the counter ion, two or more counter ions may be needed as M⁻. Further, when one carboxy group, sulfo group, or the like exists on one of the C₁₋₁₈ alkyl groups represented by R⁹ and R¹⁰ in the general formula (I), the positive charge of the quaternary nitrogen atom to which R¹⁰ binds and the anion of the carboxy group or sulfo group form an intramolecular zwitterion, and therefore the counter ion required for neutralizing the charge may become unnecessary. Furthermore, when the second cyanine compound residue has carboxy group, sulfo group, or the like in a number required for neutralizing the charge, an intramolecular zwitterion is formed with anions thereof, and therefore the counter ion required for neutralizing the charge may also become unnecessary.

An example of compound particularly preferred as the cyanine compound constituting the first cyanine compound residue is mentioned below. However, the cyanine compound constituting the first cyanine compound residue is not limited to the following specific compound. It is preferred that the carboxy group of this compound forms an amide bond or the like with a linker.

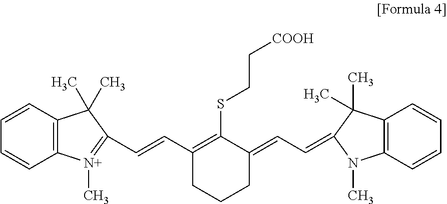

It is sufficient for the second cyanine compound residue to be substantially stable to reactive oxygen species and either equal or surpass the first cyanine compound residue functioning as a quenching group in its stability to the reactive oxygen species, and various cyanine compound residues can be used. For example, it is preferable to use a residue of a cyanine compound having a maximum fluorescence wavelength in the near infrared region, preferably a maximum fluorescence wavelength of 650 nm or larger, and showing a fluorescence quantum yield of 0.03 or larger, and it is particularly preferable to use such a residue having the following partial structure: —CH═CH—CH═CH—CH═ in the fluorophore.

As the residue of the second cyanine compound, for example, a residue of a cyanine compound represented by the following general formula (II):

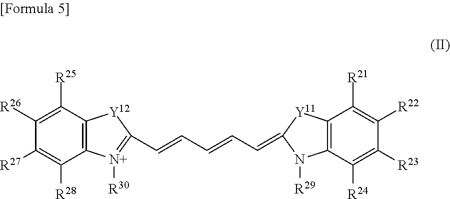

[In the formula, R²¹, R²², R²³, R²⁴, R²⁵, R²⁶, R²⁷, and R²⁸ independently represent hydrogen atom, sulfo group, phospho group, a halogen atom, or a C₁₋₆ alkyl group which may have a substituent; R²⁹ and R³⁰ independently represent a C₁₋₁₈ alkyl group which may have a substituent; and Y¹¹ and Y¹² independently represent —O—, —S—, or —C(R³¹)(R³²)— (wherein R³¹ and R³² independently represent a C₁₋₆ alkyl group which may have a substituent)] is preferred.

As the C₁₋₆ alkyl group represented by R²¹, R²², R²³, R²⁴, R²⁵, R²⁶, R²⁷, or R²⁸, methyl group, ethyl group, and the like are preferred, and as the halogen atom represented by R²¹, R²², R²³, R²⁴, R²⁵, R²⁶, R²⁷, or R²⁸, fluorine atom, chlorine atom, and the like are preferred. The sulfo group and phospho group represented by R²¹, R²², R²³, R²⁴, R²⁵, R²⁶, R²⁷, or R²⁸ may form an ester. All of R²¹, R²², R²³, R²⁴, R²⁵, R²⁶, R²⁷, and R²⁸ may represent hydrogen atom. It is preferred that one of R²¹, R²², R²³, and R²⁴ is an electron-withdrawing group such as sulfo group (except for nitro group), or one of R²⁵, R²⁶, R²⁷, and R²⁸ is an electron-withdrawing group such as sulfo group (except for nitro group), it is more preferred that one of R²¹, R²², R²³, and R²⁴ is an electron-withdrawing group such as sulfo group (except for nitro group), and one of R²⁵, R²⁶, R²⁷, and R²⁸ is an electron-withdrawing group such as sulfo group (except for nitro group), and it is particularly preferred that both R²² and R²⁶ are sulfo groups. In such case, there is obtained an effect that oxidation potential of the second cyanine compound residue increases, and stability thereof to reactive oxygen species increases.

R²⁹ and R³⁰ independently represent a C₁₋₁₈ alkyl group which may have a substituent. Examples of the alkyl group include, for example, methyl group, ethyl group, n-propyl group, isopropyl group, n-butyl group, isobutyl group, sec-butyl group, tert-butyl group, n-pentyl group, isopentyl group, neopentyl group, tert-pentyl group, 1-methylbutyl group, 2-methylbutyl group, 1-ethylpropyl group, n-hexyl group, 1-methylpentyl group, 2-methylpentyl group, 3-methylpentyl group, 4-methylpentyl group, 2,3-dimethylbutyl group, 1,3-dimethylbutyl group, 1,2-dimethylbutyl group, 1-ethylbutyl group, 2-ethylbutyl group, 1-isopropylpropyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, and the like. As the alkyl group, a linear alkyl group is preferred. Examples of the substituent that can exist on the C₁₋₁₈ alkyl group represented by R²⁹ or R³⁰ include, for example, an alkoxy group, an aryl group, a halogen atom (it may be any of fluorine atom, chlorine atom, bromine atom, and iodine atom), hydroxy group, amino group, nitro group, carboxy group or an ester thereof, sulfo group or an ester thereof, and the like. Among them, carboxy group, sulfo group, amino group, and the like are preferred, and carboxy group and sulfo group are particularly preferred. Both of R²⁹ and R³⁰ may represent an unsubstituted C₁₋₁₈ alkyl group, and it is also preferred that one of the C₁₋₁₈ alkyl groups has a substituent. It is preferred that carboxy group or sulfo group substituting for R²⁹ or R³⁰ binds with a linker. Although bonding scheme with the linker is not particularly limited, examples include, for example, an amide bond, an ester bond, a sulfoamide bond, and the like. Carboxy group or sulfo group substituting for R²⁹ or R³⁹ may directly bind to —Z—R¹¹ (R¹¹ represents hydrogen atom) of the first cyanine compound residue with an amide bond, an ester bond, a thioester bond, a sulfoamide bond, or the like without via any linker, or carboxy group, sulfo group or amino group substituting for R²⁹ or R³⁰ may directly bind to carboxy group, sulfo group or amino group substituting for a C₁₋₁₈ alkyl group which may have a substituent represented by R⁹, R¹⁰, or R¹¹ with an amide bond, an ester bond, a sulfoamide bond, or the like. Y¹¹ and Y¹² independently represent —O—, —S—, or —C(R³¹)(R³²)—, and R³¹ and R³² independently represent a C₁₋₆ alkyl group which may have a substituent. It is preferred that Y¹¹ and Y¹² represent —C(R³¹)(R³²)—, and as R³¹ and R³², methyl group is preferred.

As a particularly preferred example of the cyanine compound constituting the residue of the second cyanine compound, the following compound can be mentioned. However, the cyanine compound constituting the second cyanine compound residue is not limited to the following example. A residue obtained by removing one hydrogen atom from one of two carboxylic acids of this compound is preferred, and it is more preferred that the carboxylic acid binds to a linker with an amide bond.

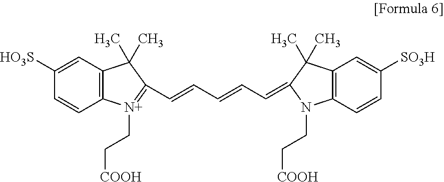

The linker is chosen so that the first cyanine compound residue can act as a quenching group for the second cyanine compound residue. So long as a linker having such a property is chosen, type of the linker is not particularly limited. The linker may be a linker consisting only of carbon atoms, or a linker containing one or two or more heteroatoms such as nitrogen atom, sulfur atom, and oxygen atom. The linker may be a linear, branched or cyclic linker, or a combination thereof. Linking atom number of the linker is, for example, about 1 to 10, preferably about 4 to 10. In this specification, the linking atom number of the linker means a number of atoms in the shortest path from the atom of one end of the linker to the atom of the other end. The linker may have one or two or more substituents. For example, the following linker can be mentioned as an example of the linker, and the linking atom number of this linker is 6.

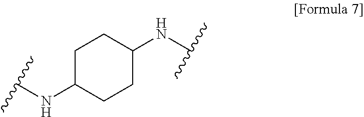

Whether the first cyanine compound residue acts as a quenching group for the second cyanine compound residue can be predicted by, for example, choosing a cyanine compound residue showing an absorption spectrum sufficiently overlapping with the fluorescence spectrum of the second cyanine compound residue as the first cyanine compound residue, measuring fluorescence quantum yields of the first cyanine compound residue and the second cyanine compound residue, and comparing them, and it is preferred that the fluorescence quantum yield of the first cyanine compound residue is ¼ or less of the fluorescence quantum yield of the second cyanine compound residue.

So long as a cyanine compound residue having an absorption spectrum sufficiently overlapping with the fluorescence spectrum of the second cyanine compound residue is chosen as the first cyanine compound residue so that FRET can efficiently occur from the second cyanine compound residue to the first cyanine compound residue, the first cyanine compound residue is not limited to a quenching group, and the residue may be a fluorophore having a substantially high fluorescence quantum yield (in this specification, the “quenching group” as the first cyanine compound residue also includes a fluorophore which efficiently emits fluorescence by FRET from the second cyanine compound residue). In this case, when the reagent for measurement of reactive oxygen of the present invention is excited at the maximum absorption wavelength of the second cyanine compound residue, fluorescence emitted by FRET from the first cyanine compound residue is observed before a reaction with reactive oxygen species, and after the reaction with reactive oxygen species, fluorescence from the second cyanine compound residue is observed, because the first cyanine compound residue is decomposed by the reactive oxygen species, and hence FRET does not occur. Therefore, the reagent can also be used as a reagent for measuring reactive oxygen species as a single wavelength excitation/double wavelength fluorescence measurement type FRET fluorescent probe.

It is sufficient that the combination of the first cyanine compound residue that functions as a quenching group, and the second cyanine compound residue is a combination in which the first cyanine compound residue either equals or surpasses the second cyanine compound residue in its reactivity to a reactive oxygen species, in other words, a combination in which the second cyanine compound residue either equals or surpasses the first cyanine compound residue in its stability to the reactive oxygen species. In carbocyanine compounds such as indocarbocyanine compounds, a longer conjugated polymethine chain in the compounds provides a lower oxidation potential and higher reactivity to reactive oxygen species. Therefore, the combination of the first cyanine compound residue and the second cyanine compound residue is preferably, for example, a combination of a dicarbocyanine compound and a dicarbocyanine compound, a tricarbocyanine compound and a tricarbocyanine compound, or a tricarbocyanine compound and a dicarbocyanine compound.

TABLE 1

| Dye | Ep (V vs SCE) |
|---|---|
| Cy5 | 0.516 |
| Cy7 | 0.476 |
| Compound 2 | 0.658 |
| Compound 3 | 0.333 |

* Values measured by using a saturated caromel electrode (SCE) as a reference electrode are shown.

One or two of R¹ to R¹⁰ in the formula (I) or R²¹ to R³⁰ in the formula (II) may be a group which can be buried in a cell membrane. In that case, the reagent of the present invention can be used as a membrane localizing type fluorescent probe to efficiently measure reactive oxygen species generated around cell membranes. As the group which can be buried in a cell membrane, a linear or branched C₇₋₁₈ alkyl group and a phospholipid are preferred (for example, phosphatidylethanolamines, phosphatidylcholines, phosphatidylserines, phosphatidylinositols, phosphatidylglycerols, cardiolipins, sphingomyelins, ceramide phosphorylethanolamines, ceramide phosphorylglycerols, ceramide phosphoryl glycerol phosphates, 1,2-dimyristoyl-1,2-deoxyphosphatidykholines, plasmalogens, and phosphatidic acids, however, the aliphatic acid residue in these phospholipids is not particularly limited, and phospholipids having one or two saturated or unsaturated aliphatic acid residues having about 12 to 20 carbon atoms can be used).

When the reagent of the present invention is used in cells or biological tissues, or in vivo, by appropriately choosing groups substituting for R¹ to R¹⁰ in the formula (I) and R²¹ to R³⁰ in the formula (II) or the substituents of the alkyl groups which may have a substituent as R¹ to R¹⁰ in the formula (I) and R²¹ to R³⁰ in the formula (II) to control water-solubility of the reagent of the present invention, the reagent can be used as a membrane permeable type or non-membrane permeable type probe. For example, a compound of the present invention having one or two, and preferably three or more, of sulfo groups or carboxy groups has extremely high water-solubility and non-membrane permeability, and therefore the compound is not taken up into cells. Therefore, such compound can be preferably used to detect reactive oxygen species released out of cells. Further, for example, by incorporating one or two chains of polyalkylene glycol such as polyethylene glycol and polypropylene glycol as substituents, desired water-solubility can be appropriately imparted to the reagent of the present invention depending on the number of introduced polyalkylene glycol substituents and polyalkylene glycol chain length.

The reagent of the present invention may exist as a hydrate or solvate, and these substances also fall within the scope of the present invention. The reagent of the present invention may have one or more asymmetric carbons depending on types of substituents, and stereoisomers such as optically active substances based on one or two or more asymmetric carbons and diastereomers based on two or more asymmetric carbons, as well as arbitrary mixtures of stereoisomers, racemates, and the like all fall within the scope of the present invention.

Preparation methods of typical compounds as the reagent of the present invention are specifically shown in Examples of the specification. Therefore, those skilled in the art can readily prepare the reagent of the present invention on the basis of these explanations by appropriately choosing starting materials, reaction conditions, reagents, and the like, and modifying or altering the methods as required.

The term “measurement” used in this specification should be construed in the broadest sense thereof, including quantitative and qualitative measurements, as well as measurement, investigation, detection and the like carried out for the purpose of diagnosis or the like. The method for measurement of reactive oxygen species of the present invention generally comprises (A) the step of reacting the aforementioned reagent and a reactive oxygen species, and (B) the step of measuring fluorescence of a decomposition product of the aforementioned reagent produced in the aforementioned step (A). Examples of reactive oxygen species measurable with the reagent of the present invention include hydroxyl radical, peroxynitrite, hypochlorite ion, nitrogen monoxide, hydrogen peroxide, superoxide anion, singlet oxygen, and the like.

When the reagent of the present invention is used, although means for measuring fluorescence is not particularly limited, a method of measuring fluorescence spectrum in vitro, a method of measuring fluorescence spectrum in vivo by using a bioimaging technique and the like may be employed. For example, when quantification is carried out, it is desirable to prepare a calibration curve beforehand according to a conventional method. As a quantitative hydroxyl radical generation system, for example, a gamma-radiolysis method and the like can be used. As a singlet oxygen generation system, for example, the naphthalene endoperoxide system (Saito, I, et. al., J. Am. Chem. Soc., 107, pp. 6329-6334, 1985) and the like can be used. If the reagent of the present invention is incorporated into cells by microinjection or the like, reactive oxygen species localizing in individual cells can be measured in real time with high sensitivity by a bioimaging technique, and if the reagent is used in culture broth for cell or tissue sections, or in a perfusate, reactive oxygen species released from the cells or biological tissues can be measured. By using the reagent of the present invention, oxidation stress in cells or, biological tissues can be measured in real time, and thus the reagent can be preferably used for cause investigation of disease pathologies, development of therapeutic agents, and the like.

The reagent of the present invention may also be used as a composition formulated with additives ordinarily used for preparation of reagents, if desired. For example, as additives for use of the reagent in a physiological condition, additives such as dissolving aids, pH modifiers, buffers, isotonic agents and the like can be used, and amounts of these additives can suitably be chosen by those skilled in the art. The compositions may be provided as compositions in appropriate forms, for example, powdery mixtures, lyophilized products, granules, tablets, solutions and the like.

### EXAMPLES

The present invention will be more specifically explained with reference to examples. However, the scope of the present invention is not limited to the following examples.

### Example 1

### Preparation of Reagents for Measurement of Reactive Oxygen of the Present Invention

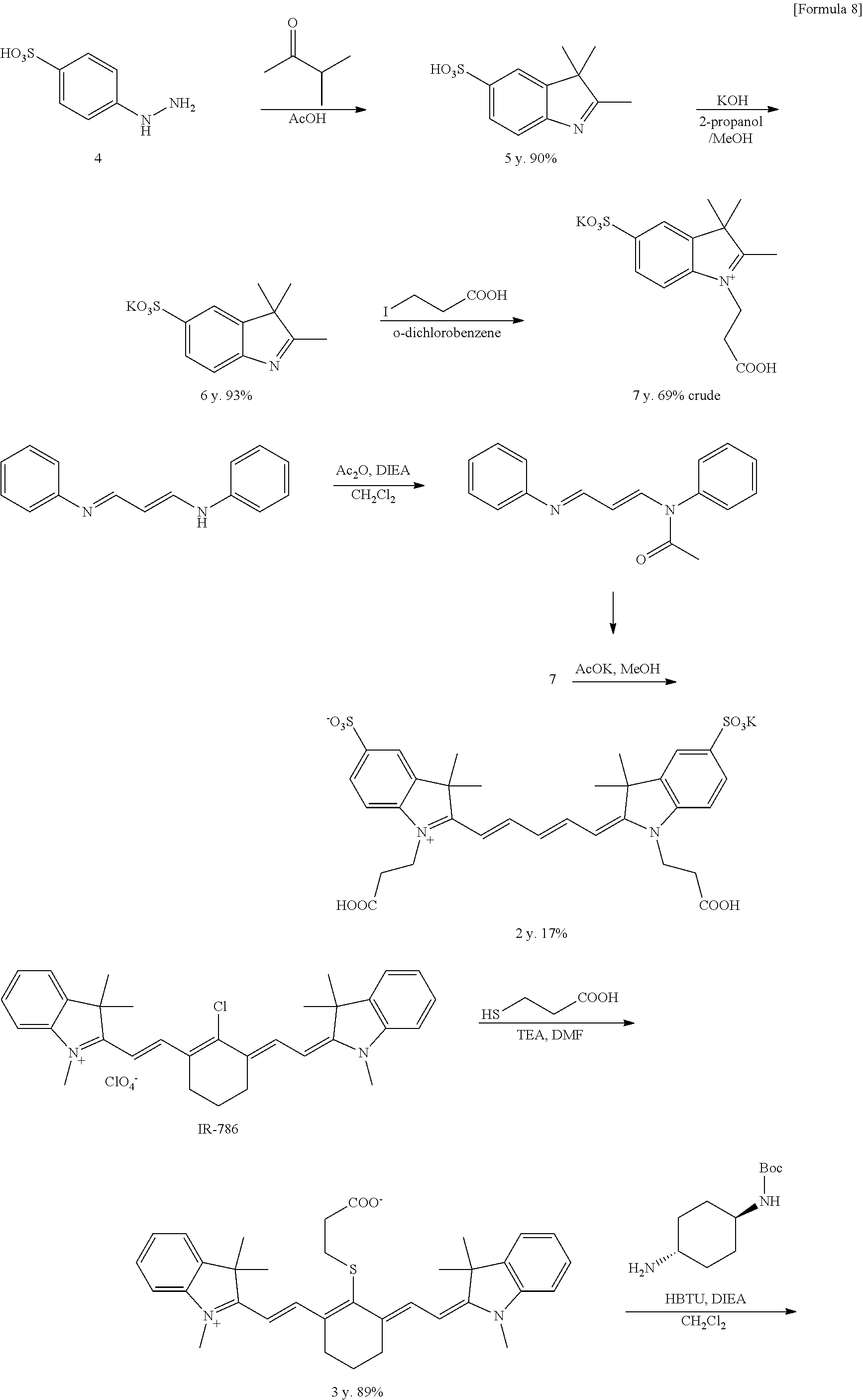

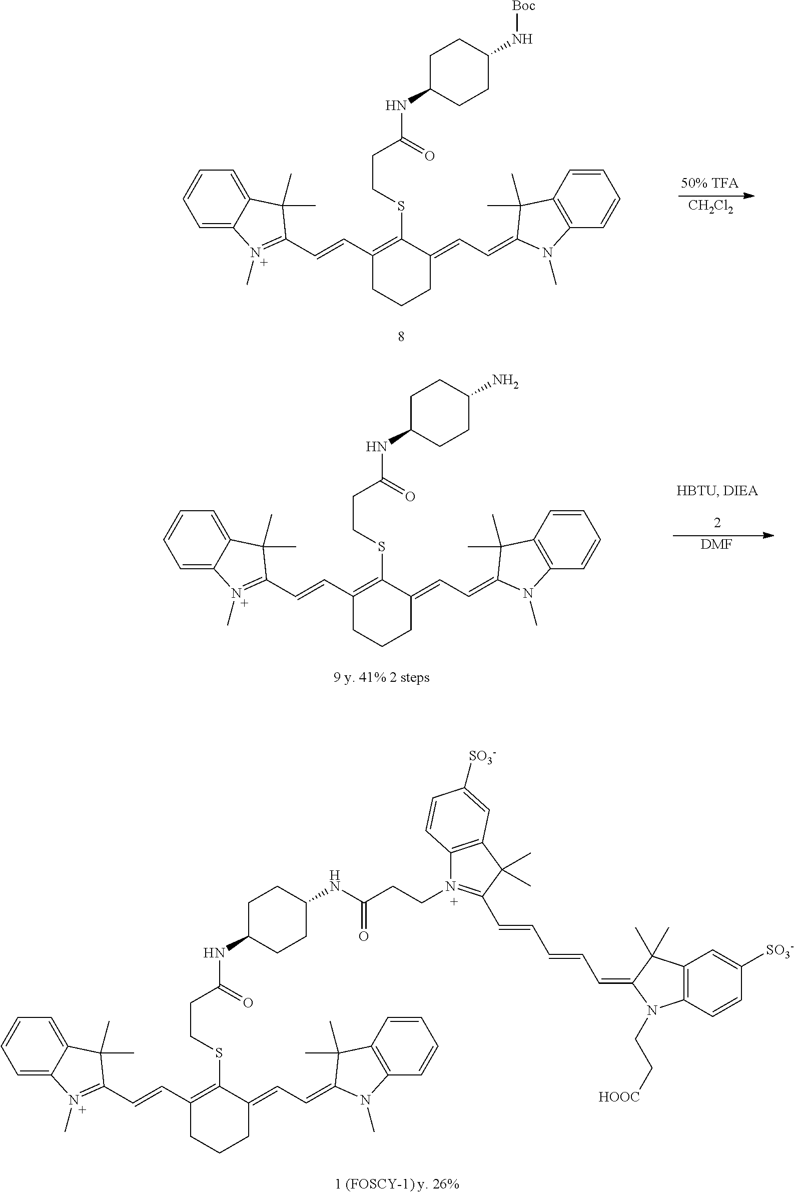

### (1) Compound 5

Hydrazinobenzenesulfonic acid 4 (12.9 g, 67 mmol) and 3-methyl-2-butanone (7 mL, 67 mmol) were dissolved in acetic acid (30 mL), and the solution was refluxed by heating for 14 hours with stirring. The solution was left to cool to room temperature, and the precipitates collected by filtration of the solution were washed with diethyl ether to obtain the objective substance (18.0 g).

### (2) Compound 6

Compound 5 (18.0 g, 59 mmol) was dissolved in methanol (20 mL), a saturated solution of potassium hydroxide in isopropyl alcohol (300 mL) was added to the solution, and the mixture was stirred. The yellow precipitates collected by filtration of the mixture were washed with isopropyl alcohol to obtain the objective substance (15.2 g).

### (3) Compound 7

Compound 6 (30.5 g, 0.11 mol) and 3-iodopropionic acid (25.0 g, 0.13 mol) were dissolved in o-dichlorobenzene (150 mL), and the solution was heated at 110° C. for 19 hours with stirring. The solution was left to cool to room temperature, then the supernatant was discarded, and the residue was washed with isopropyl alcohol and diethyl ether to obtain the objective substance (26.5 g).

### (4) Compound 2

Malonaldehyde dianilide hydrochloride (2.5 g, 9.8 mmol) was dissolved in a mixture of methylene chloride (15 mL) and N,N-diisopropylethylamine (1.5 mL). A mixture of acetic anhydride (1.5 mL) and methylene chloride (5 mL) was added dropwise to the solution with stirring at room temperature, and the mixture was further stirred at room temperature for 4 hours. A solution of Compound 7 (6.8 g, 19 mmol) and potassium acetate (1.0 g, 10 mmol) in methanol (20 mL) was refluxed by heating, and the yellow solution obtained above was added dropwise to the solution. The mixture was further heated for 10 hours, and left to cool to room temperature, and then the precipitates obtained by filtration of the mixture were washed with isopropyl alcohol and diethyl ether and purified by column chromatography using reverse phase silica gel to obtain the objective substance (1.1 g).

### (5) Compound 3

IR-786 perchlorate (CAS No. 115970-66-6, 1.5 g, 2.6 mmol) was dissolved in dimethylformamide (DMF, 10 mL), 3-mercaptopropionic acid (265 μL, 3.0 mmol) and triethylamine (425 μL, 3.0 mmol) was added to the solution, and the mixture was stirred at room temperature for 20 hours. Methylene chloride was added to the reaction mixture, and the resulting mixture was subjected to extraction with methylene chloride/saturated brine. The organic layer was collected, dried over sodium sulfate, and filtered, and then the solvent was evaporated. The residue was recrystallized from isopropyl alcohol to obtain the objective substance (1.3 g).

### (6) Compound 8

Compound 3 (217 mg, 0.39 mmol) and O-(benzotriazol-1-yl) -N,N,N′,N′-tetramethyluronium hexafluorophosphate (HBTU, 173 mg, 0.46 mmol) were dissolved in methylene chloride (10 mL), and N-tert-butoxycarbonyl-trans-1,4-cyclohexanediamine (98 mg, 0.46 mmol) and N,N-diisopropylethylamine (75 μL) was further added to the solution. The reaction mixture was stirred at room temperature for 4 hours, and then methylene chloride was added, and the mixture was subjected to extraction with methylene chloride/saturated aqueous sodium hydrogencarbonate. The organic layer was collected, dried over sodium sulfate, and filtered, and then the solvent was evaporated. This compound was used for the next reaction without purification.

### (7) Compound 9

Compound 8 was dissolved in a 50% solution of trifluoroacetic acid in methylene chloride (20 mL), and the solution was stirred at room temperature for 3 hours. The solvent was evaporated, the residue was dissolved in a small volume of methanol, and then diethyl ether (about 200 mL) was added to the solution to reprecipitate the residue. The precipitates obtained by filtration of the mixture were recrystallized from isopropyl alcohol to obtain the objective substance (104 mg).

### (8) Compound 1 (FOSCY-1)

Compound 2 (233 mg, 0.33 mmol) was dissolved in dimethylformamide (10 mL), a solution of HBTU (108 mg, 0.28 mmol) in dimethylformamide (10 mL) was added dropwise, and then a solution of Compound 9 (80 mg, 0.12 mmol) and N,N-diisopropylethylamine (25 μL) in dimethylformamide (10 mL) to the solution. The mixture was stirred at room temperature for 9 hours, and then the solvent was evaporated. The resulting residue was purified by preparative HPLC to obtain the objective substance (40 mg).

¹H NMR (300 MHz, DMF-d₇): δ 8.77 (d, 2H, J=14.1 Hz), 8.47 (m, 2H), 8.33 (br, 1H), 7.85-7.26 (m, 15H), 6.64-6.53 (m, 2H), 6.37 (m, 3H), 4.42 (br, 4H), 3.78 (s, 6H), 3.04 (t, 2H, J=7.2 Hz), 2.63 (m, 4H), 2.46 (t, 2H, J=7.2 Hz), 1.83-1.68 (m, 30H), 1.20-1.07 (m, 4H).

¹³C-NMR (100 MHz, DMF-d₇): δ 179.6, 178.9, 178.4, 177.8, 174.6, 174.5, 168.2, 167.9, 161.0, 160.0, 151.8, 151.6, 150.7, 149.0, 147.8, 147.7, 146.8, 146.5, 146.4, 138.9, 134.2, 131.9, 131.8, 130.5, 128.0, 125.6, 125.5, 116.7, 116.1, 115.8, 110.1, 109.5, 107.2, 54.9, 54.7, 54.6, 53.2, 53.1, 51.6, 51.5, 51.2, 41.5, 38.9, 37.6, 36.7, 32.8, 32.3, 31.6, 26.5, 14.1; 14.0, 13.7.

HRMS (ESI⁻); m/z calcd for (M-H)⁻, 1287.53328. found, 1287.53710.

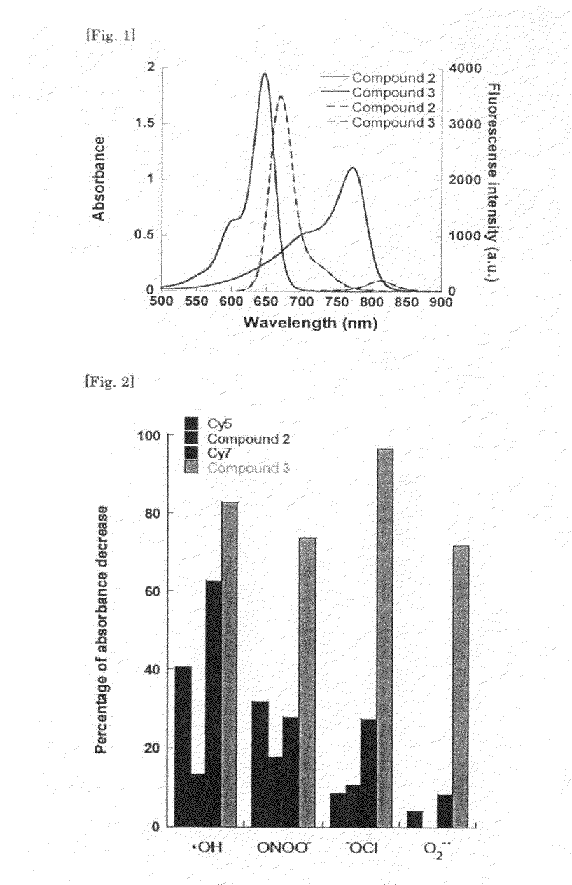

The UV spectra and fluorescence spectra of Compound 2 (cyanine compound constituting the second cyanine compound residue) and Compound 3 (cyanine compound constituting the first cyanine compound residue) obtained above are shown in FIG. 1. In the drawing, the solid lines indicate the absorption spectra, and the broken lines indicate the fluorescence spectra. As a result, it can be understood that the fluorescence spectrum of Compound 2 and the absorption spectrum of Compound 3 have a large overlapping range, and thus they constitute a combination suitable for inducing resonance energy transfer.

The photochemical characteristics of Compound 1 (FOSCY-1) were as follows.

Maximum absorption wavelength: 644 nm (in 100 mM phosphate buffer (pH 7.4))
Maximum fluorescence wavelength: 668 nm (in 100 mM phosphate buffer (pH 7.4))
Quantum yield φ: 0.014 (relative value based on the value of fluorescence standard of cresyl violet in methanol: 0.54)
Molar absorption coefficient ε (×10⁵ M⁻¹cm⁻¹): 1.5

### Example 2

Cy5, Cy7, Compound 2 (cyanine compound constituting the second cyanine compound residue) and Compound 3 (cyanine compound constituting the first cyanine compound residue), the latter two of which were obtained above, were reacted with hydroxyl radical, peroxynitrite, hypochlorite ion, and superoxide anion, and change of absorbance at the maximum absorption wavelength was measured. For the measurement, 10 μM solutions of Cy5, Cy7, Compound 2, and Compound 3 in 0.1 M phosphate buffer were prepared, and the measurement was performed with the prepared solutions under the following conditions.

### (a) Hydroxyl Radical

Hydrogen peroxide and iron(II) perchlorate were added to final concentrations of 1 mM and 50 μM, respectively.

### (b) Peroxynitrite

Peroxynitrite was added to a final concentration of 10 μM.

### (c) Hypochlorite Ion

Hypochlorite ions were added to a final concentration of 10 μM.

### (d) Superoxide Anion

Xanthine oxidase and xanthine were added to final concentrations of 4 mU and 33 μM, respectively.

The results are shown in FIG. 2. In the graph, the test results for those reactive oxygen species are indicated in the order of Cy5, Compound 2, Cy7, and Compound 3 from the left.

From the results shown in FIG. 2, it was confirmed that Cy7, which is a tricarbocyanine compound, showed larger decrease of absorbance than did Cy5, which is a dicarbocyanine compound, upon addition of all the reactive oxygen species, and thus reactivity of Cy7 to the reactive oxygen species was higher than that of Cy5. Further, it was confirmed that Compound 3, which is a derivative of Cy7 where thioether group is introduced into the conjugated polymethine chain, showed larger decrease of absorbance than did Cy7 for all the reactive oxygen species, and thus reactivity of Compound 3 to the reactive oxygen species was higher than that of Cy7. This indicated that introduction of thioether group into the conjugated polymethine chain improved reactivity of cyanine compounds to the reactive oxygen species. However, Compound 2, which is a derivative of Cy5 where electron-withdrawing sulfo group was introduced into the indolenine moiety, showed the smallest decrease of absorbance for all the reactive oxygen species, in particular, it showed no decrease of absorbance for superoxide anion. Therefore, it was demonstrated that introduction of an electron-withdrawing substituent such as sulfo group into the indolenine moiety improved stability of cyanine compounds to the reactive oxygen species.

### Example 3

The reagent for measurement of reactive oxygen of the present invention was reacted with various reactive oxygen species, and change of fluorescence spectrum was measured. The measurement was performed as follows.

### (1) Hydroxyl Radical

To a 1 μM solution of Compound 1 in a phosphate buffer (0.1 M, pH 7.4, containing 0.1% DMF as a cosolvent) vigorously stirred at room temperature in a flask, 1 M aqueous H₂O₂ was added to a final concentration of 0.1 mM, and then 1 mM aqueous iron(II) perchlorate was added dropwise to a final concentration of 0 μM, 0.13 μM, 0.25 μM, 0.5 μM, 1 μM, 2 μM, or 3 μM. After 1 minute, fluorescence spectrum obtained with an excitation light of 644 nm was measured by using a fluorophotometer.

### (2) Peroxynitrite

To a 1 μM solution of Compound 1 in a phosphate buffer (0.1 M, pH 7.4, containing 0.1% DMF as a cosolvent) stirred at room temperature in a cuvette, a 1 mM solution of peroxynitrite in 0.1 N aqueous sodium hydroxide was added dropwise to a final concentration of 0 μM, 0.3 μM, 0.7 μM, 1 μM or 2 μM. After 1 minute, fluorescence spectrum obtained with an excitation light of 644 nm was measured by using a fluorophotometer.

### (3) Hypochlorite Ion

To a 1 μM solution of Compound 1 in a phosphate buffer (0.1 M, pH 7.4, containing 0.1% DMF as a cosolvent) stirred at room temperature in a cuvette, a 1 mM solution of sodium hypochlorite in 0.1 N aqueous sodium hydroxide was added dropwise to a final concentration of 0 μM, 0.3 μM, 0.7 μM, 1 μM, 2 μM or 3 μM. After 1 minute, fluorescence spectrum obtained with an excitation light of 644 nm was measured by using a fluorophotometer.

### (4) Superoxide Anion

To a 1 μM solution of Compound 1 in a phosphate buffer (0.1 M, pH 7.4, containing 0.1% DMF as a cosolvent) stirred at room temperature in a cuvette, an aqueous solution of xanthine oxidase was added to a final concentration of 4 mU/mL, and then a solution of xanthine in DMF was added to a final concentration of 33 μM. After 30 minutes, fluorescence spectrum obtained with an excitation light of 644 nm was measured by using a fluorophotometer. When a superoxide dismutase treatment was used, an aqueous solution of superoxide dismutase was added to a final concentration of 60 U/mL before addition of the aqueous solution of xanthine oxidase.

### (5) Singlet Oxygen

To a 1 μM solution of Compound 1 in heavy water stirred at 37° C. in a cuvette, a solution of a singlet oxygen releasing agent EP-1 (3-(1,4-dihydro-1,4-epidioxy-1-naphthyl)propionic acid), which is known to heat-dependently release singlet oxygen, in DMF was added to a final concentration of 0.2 mM, and after 30 minutes, fluorescence spectrum obtained with an excitation light of 644 nm was measured by using a fluorophotometer.

### (6) Hydrogen Peroxide

To a 1 μM solution of Compound 1 in a phosphate buffer (0.1 M, pH 7.4, containing 0.1% DMF as a cosolvent) stirred at room temperature in a cuvette, 1 M aqueous H₂O₂ was added to a final concentration of 10 mM, and after 30 minutes, fluorescence spectrum obtained with an excitation light of 644 nm was measured by using a fluorophotometer.

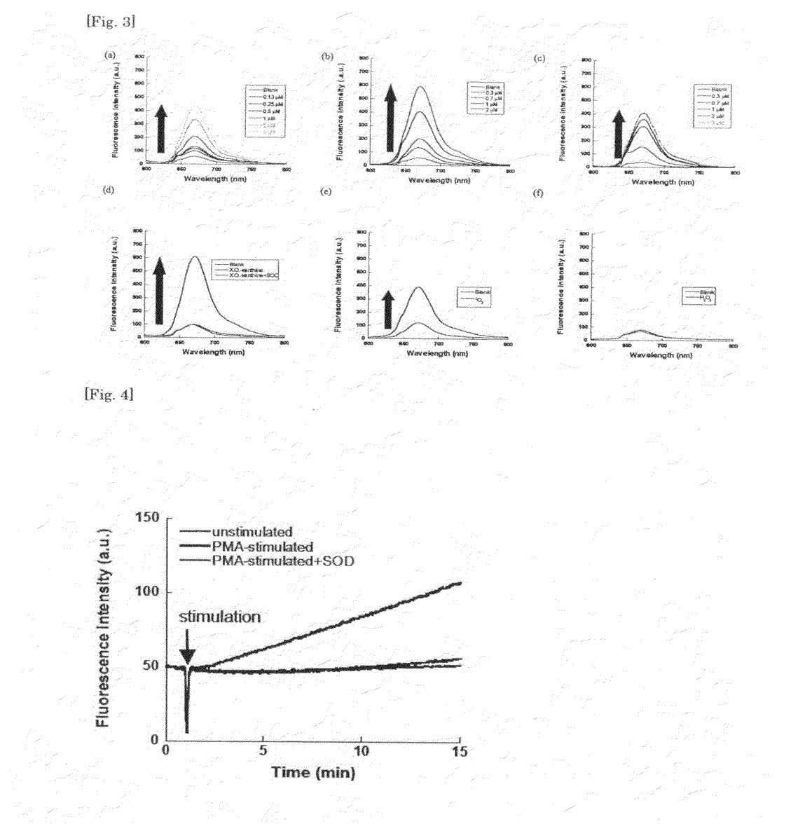

The results are shown in FIG. 3. From the results shown in FIG. 3, it can be confirmed that Compound 1 of the present invention can react with hydroxyl radical, peroxynitrite and hypochlorite ion in a concentration dependent manner to show increase of fluorescence intensity at 668 nm. Moreover, it also showed increase of fluorescence intensity at 668 nm with addition of superoxide anion or singlet oxygen, and therefore it was shown that hydroxyl radical, peroxynitrite, hypochlorite ion, superoxide anion, and singlet oxygen can be measured with Compound 1 by using an excitation light of 644 nm in the near infrared region.

### Example 4

### Measurement of Superoxide Anions Produced by HL60 Cells Derived from Human Promyelocytic Leukemia

HL60 cells cultured by using a CO₂ incubator in the Roswell Park Memorial Institute (RPMI) medium containing 10% (V/V) fetal bovine serum, penicillin (100 U/mL) and streptomycin (100 μg/mL) were diluted to 1×10⁶ cells/mL with Hanks' balanced salts solution (HESS), and 3 mL of the cell suspension was transferred into a plastic cuvette. Compound 1 was added to a final concentration of 0.1 μM (0.1% DMF was contained as a cosolvent), and the mixture was slowly stirred at 37° C. One minute after the start of the measurement, 1 μg of phorbol 12-myristate 13-acetate (PMA) (0.2% DMF was contained as a cosolvent) or 3 μL of DMF as a control was added. When a superoxide dismutase treatment was used, superoxide dismutase (SOD) was added to a final concentration of 60 U/mL before the addition of PMA. Fluorescence intensity was measured every minute at a fluorescence wavelength of 668 nm using an excitation light of 645 nm. The results are shown in FIG. 4. Marked increase of fluorescence was observed after the addition of PMA, which showed superoxide anions were generated by the HL60 cells and released out of the cells. When SOD was added to the measurement mixture beforehand, the increase of fluorescence was suppressed, by which the reactive oxygen species were confirmed to be superoxide anions. As described above, if the reagent for measurement of reactive oxygen of the present invention is used, reactive oxygen species produced by live cells can be measured with good sensitivity.

1. A reagent for measurement of reactive oxygen containing a compound comprising a first cyanine compound residue and a second cyanine compound residue bound to each other and having the following characteristics features (i) to (iii):
(i) the first cyanine compound residue and the second cyanine compound residue are directly bound to each other via substituents on the first cyanine compound residue and the second cyanine compound residue, or the first cyanine compound residue and the second cyanine compound residue are bound via a linker,
(ii) the first cyanine compound residue has a property that it easily reacts with a reactive oxygen species and is thereby decomposed, and
(iii) the second cyanine compound residue either equals or surpasses the first cyanine compound residue in its stability to the reactive oxygen species, and the first cyanine compound residue has a property that it acts as a quenching group for the second cyanine compound residue.

2. The reagent according to claim 1, wherein —S— group substitutes for one carbon atom in a conjugated polymethine chain of the first cyanine compound residue.

3. The reagent according to claim 1, wherein the second cyanine compound residue has one or two sulfo groups in a nitrogen-containing heterocyclic moiety.

4. The reagent according to claim 1, wherein the first cyanine compound residue has the following partial substructure in the fluorophore:

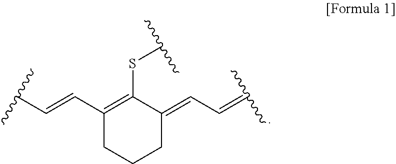

5. The reagent according to claim 1, wherein the second cyanine compound residue has a maximum fluorescence wavelength in the near infrared region, and shows a fluorescence quantum yield of 0.03 or larger.

6. The reagent according to claim 1, wherein the first cyanine compound residue and the second cyanine compound residue are bound via a linker.

7. The reagent according to claim 6, wherein the second cyanine compound residue binds to the linker with carboxy group or sulfo group.

8. The reagent according to claim 1, wherein the first cyanine compound residue and the second cyanine compound residue are tetramethylindocarbocyanine compound residues.

9. A fluorescent probe for measurement of a reactive oxygen represented by the following formula:

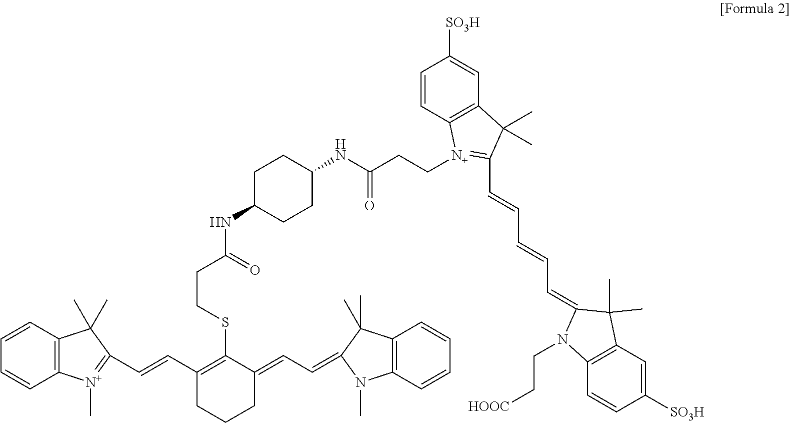

10. A method for measurement of a reactive oxygen species comprising the following steps: (A) reacting the reagent according to claim 1 and a reactive oxygen species, and (B) measuring fluorescence of a decomposition product of the reagent according to claim 1 produced in the aforementioned step (A).
